(19) United States
(12) Patent Application Publication
(10) Pub. No.: US 2001/0039263 A1
(43) Pub. Date: Nov. 8, 2001

(54) Chimeric oligonucleotides and the use thereof

(75) Inventors: Eckart Matthes, Eggersdorf (DE); Martin Von Janta-Lipinski, Berlin (DE)

Correspondence Address: BRUCE LONDA, NORRIS, MCLAUGHLIN & MARCUS, P.A., 220 EAST 42ND STREET, 30TH FLOOR, NEW YORK, NY, 10017, US

(73) Assignee: Max-Delbruck-Centrum fur Molekulare Medizin

(21) Appl. No.: 09/817,387

(22) Filed: Mar. 26, 2001

Related U.S. Application Data
(63) Continuation-in-part of application No. 09/423,157, filed on Feb. 18, 2000, now abandoned.
Application No. 09/423,157 is a 371 of international application No. PCT/DE98/01216, filed on May 4, 1998.

(30) Foreign Application Priority Data
May 2, 1997 (DE) 197 20 151.2

(51) Int. Cl. A61K 48/00; C07H 21/02; C07H 21/04

(52) U.S. Cl. 514/44; 536/23.1

(57) ABSTRACT

The invention relates to novel chimeric oligonucleotides and their use, especially to bind and inhibit the enzyme telomerase. The oligonucleotides were prepared in view of RNA's non-binding activity to phosphorothioates and the primer binding site of the enzyme. The oligonucleotides bind to the primer binding site thereby resulting in maximum inhibition of the enzyme.

This application is a continuation-in-part application of U.S. Ser. No. 09/423,157 filed Feb. 18, 2000, now abandoned.

### BACKGROUND OF THE INVENTION

The invention relates to novel chimeric oligonucleotides and their use, especially to inhibit the enzyme telomerase and to produce pharmaceutical formulations used in antitumoral therapy. Thus, the invention relates also to drugs which contain the chimeric oligonucleotides.

The present cancer chemotherapy applied in clinics has to be regarded as completely insufficient. Only in the case of a few tumours it results in healing, the majority of malignant tumours has to be regarded as not being curable by means of the present therapy. It is mainly a non-specific antiproliferative therapy, i.e. aimed at inhibiting the growth and cell division. This effect is not restricted to tumour cells but relates also to a number of renewable tissues with a high rate of cell division such as e.g. bone marrow, intestinal and skin epithelial cells which explains also the strong cytotoxic side effects.

The findings that mutations in a multitude of oncogens and repressor genes of the cells are causally related with the development of tumours, have resulted in a multitude of efforts made to develop selective, cause-oriented chemotherapeutic agents. This involves e.g. inhibitors of farnesyltransferase and tyrosinekinase inhibitors, gene therapies aimed to restore suppressor gene functions or DNA repair or antisense oligonucleotides against various oncogens (e.g. ras, raf, erb). These new cancer targets promising a higher selectivity and efficiency includes also telomerase.

Telomerase is a RNA dependent DNA polymerase elongating the extreme 3′-ends of chromosomal DNA. Thereby, it uses a small region of the RNA which is an integral part of the enzyme as a template for the synthesis of hexanucleotide repeats. This so-called telomeric DNA has the sequence TTAGGG in man.

Its function consists, on the one hand, in protecting chromosome ends against the degradation or fusion—preventing karyotypical changes and genetic instabilities, on the other hand, in counting the number of running cell divisions. The length of telemeric DNA was found to be between about 1000 and 12000 base pours (Harley, 1991).

This heterogeneity of the telomere length might be explained by two mechanisms. On the one hand, a loss of telomeric DNA is connected with each round of DNA replication and thus with each cell division, on the other hand, by the activity of the telomerase which can compensate for this loss in specific cells and under specific conditions.

Without the possibility of compensation the loss of the telomeric DNA reaches finally a critical lower limit (about 7000 Bp; Bacchetti, 1996) which is considered as a signal for the cell to induce proliferation stop and cellular senescence. Therefore the length of telomeric DNA is considered as a “mitotic clock” counting the number of all divisions.

Thus, this mechanism may explain that the length of the telomeric DNA declines strongly with the age in most of the somatic cells. Only the so-called immortalised cells involving e.g. germ cells and fetal cells express telomerase which replaces the loss of telemeric DNA and giving them a nearly unlimited proliferation capacity (Harley, 1991).

In 1994, for the first time, telomerase activities were detected in tumour cells of a human ovarial carcinoma and in cultivated human tumour cells (Counter et al., 1994). Since this discovery telomerase could be detected in nearly all human tumors. This was possible mainly by developing an PCR-based telomerase (telomere repeat amplification protocol, TRAP) which allowing an increase of the sensitivity of the test by about 10⁴ times, making the telomerase activity detectable only in few cells (about 50 to 100) (Kim et al.; Piatyszek et al. 1995).

Depending on the type and stage of the tumours examined it was possible to detect a telomerase activity in 80-95% of them (Healy, 1995; Autexier et al. 1996; Shay et al., 1996; Hiyama et al., 1997). Obviously, the unlimited proliferation potential of tumour cells is dependent on the expression of telomerase. Thus, this enzyme might be considered as a new important target for cancer therapy (Healy, 1995; Holt et al., 1996; Hamilton et al., 1996).

Besides tumour cells also germ cells as immortal cells express this enzyme. In addition, in the last two years low telomerase activities have been detected in stem cells of renewable tissues (e.g. of skin, intestine and bone marrow), and in leucocytes and lymphocytes, when activated (Counter et al., 1995).

So far it is not clear what the function of the telomerase is in these stem cells and whether telomerase is used really to elongate telomeres.

A therapy directed to inhibit the telomerase activity might have few side effects, excluding human germ cells however. In contrast, stem cells of renewable tissues have longer telomeres than cancer cells and have a lower proliferation rate than cancer cells, which both might protect them against telomere shortening induced by telemerase inhibitors (Holt et al., 1996). Thus, such an antitelomerase therapy may be regarded as an efficient and selective therapy of malignant tumours which is superior to the present chemotherapy.

Some modified nucleoside triphosphates have been examined as potential inhibitors of human telomerase. Most of these compounds were developed earlier as inhibitors of the reverse transcriptase of HIV (human immunodeficiency virus). Both, retroviral reverse transcriptases and telomerase use a RNA as template for the synthesis of DNA. This functional similarity might be the basis for findings that 2′, 3′ dideoxyguanosine triphosphate (ddGTP), guanine arabinosyl triphosphate (araGTP), 2′, 3′ dideoxythymidine triphosphate (ddTTP), 2′, 3′ didehydro 2′, 3′ dideoxythymidine triphosphate (ddeTTP) and 3′azidothymidine triphosphate (AzTTTP) are also inhibitors of telomerase.

As nucleosides only azidothymidine (AzT) and 2′, 3′ dideoxyguanosine (ddG) resulted in a shortening of the telomeric DNA in some cell lines, when applied for a linger time, however, without changing essentially their growth behaviour or inducing a proliferation stop (Strahl et al., 1996).

Furthermore, the telomerase RNA tighty bond to the telomerase Protein was described as another promising target. Thus, antisense oligonucleotides binding complementary to the template region of RNA inhibit the enzyme activity. Indeed, it was shown that permanent inhibition of telomerase in HeLa cells expressing antisense oligonucleotides against template RNA of telomerase caused an increasing shortening of the telomeric DNA which resulted in all death after 23-26 population doublings (Feng et al., 1995).

Antisense oligomers in which the sugar phosphate backbone is replaced by N(2-amino ethyl) glycine (peptide nucleic acids, PNA) were described to inhibit the telomerase in vitro at nanomolecular range (Norton et al., 1996). Here agein, the template region of telomerase RNA was used as target. However, it is also known from these excellently binding PNAs that they, could not be taken up by cell membranes whicvh limits their applicability (Hanvey et al., 1992). In the same paper Norton et al. reported that oligonucleotides modified by phosphorothioates are efficient, but non-specific inhibitors of telomerase.

That means, there has to be stated that, for the time being, an efficient inhibitor of telomerase selectively applicable to tumour cells is not available.

### SUMMARY OF THE INVENTION

Therefore, the invention was based on the task to develop selective and highly efficient inhibitors of human telomerase which may inhibit this enzyme selectively also in cells over a long period.

The invention is implemented according to the claims and based on the surprising finding that the phosphorothioates described do not bind to RNA but sequence-non-specifically to a protein site, called primer binding site which is thougt to fix the end of telomeric DNA to be elongated. That means, there exist two neighboring targets for telomerase inhibition.

### DESCRIPTION OF THE PREFERRED EMBODIMENTS

The invention is implemented according to the claims and based on the surprising finding that the phosphorothioates described do not bind to RNA but sequence-non-specifically to a protein site, called primer binding site which is thougt to fix the end of telomeric DNA to be elongated. That means, there exist two neighboring targets for telomerase inhibition.

According to the invention these two targets—first RNA and secondly the primer binding site of the protein—are blocked by oligonucleotides, thus allowing a therapeutically optimum inhibition.

According to the invention such chimeric oligonucleotides were prepared consisting of variously modified oligomeres optimized in view of the two targets and block, at the same time, the two enzyme binding sites of the telomeric DNA. These two differently modified parts of the oligonucleotide are linked together.

They proved to be more efficient and selective than their individual components. In particular, chimeric oligonucleotides have proved to be successful which are modified at the 5′ end of the oligonucleotide by phosphorothioates, thus binding to the protein whereas being extended at the 3′ end, e.g. by phosphoamidates or, if necessary, via a linker by PNAs thus concerning telomerase RNA. In this way selectivity and efficiency of phosphorothioate-modified oligonucleotides is increased essentially. In addition, we found that a further, remarkable increase in efficiency may be reached if the 3′ end of the chimeric oligonucleotides according to the invention is modified by such nucleosides which additionally inhibit the catalytic centre of the enzyme (e.g. 3′azidodeoxyguanosine).

According to the invention chimeric oligonucleotides of the general formula I are characterized by the following structures:

Formula I is a combination of formulas II and III/1, III/2 and III/3

Formula II represents the oligomer binding to the primer binding site of telomerase

Formula III represents the oligomer binding to RNA.

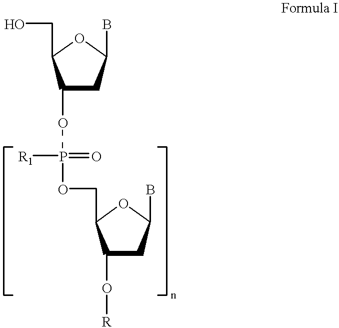

wherein R is selected from the group consisting of

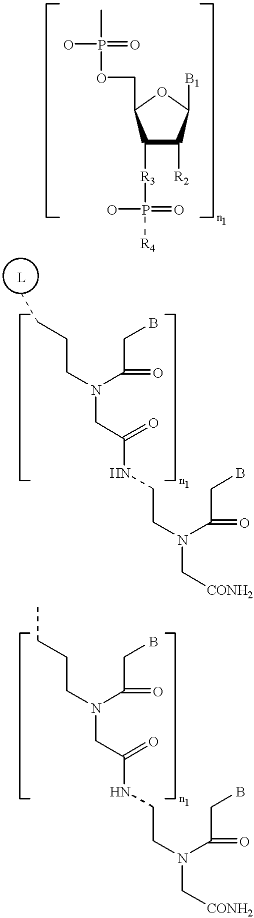

wherein

n is at least 10 and not more than 20,

R₁ is selected from the group consisting of S⁻, CH₃, and O⁻,

B is selected from the group consisting of thymine, cytosine, adenine, and guanine,

n₁ is at least 3 and not more than 17,

B₁ is selected from the group consisting of thymine, cytosine, adenine, guanine, 5-propyluracil, and 5-propylcytosine,

R₂ is selected from the group consisting of H, F, NH₂, O-alkyl (C₁-C₅), O-allyl, and O-methoxyethoxy,

R₃ is selected from the group consisting of NH and O, wherein if R₃ is NH, R₂ must not be selected from the group consisting of NH₂, O-alkyl (C₁-C₅), O-allyl, and O-methoxyethoxy,

R₄ is selected from the group consisting of 2′,3′-dideoxy-3′-fluoroguanosine, 2′,3′-dideoxy-3′-azidoguanosine, 2′,3′-dideoxy-3′-aminoguanosine, 2′,3′-epoxyguanosine, acyclovir, gancyclovir, 2′-deoxyadenosine, 2′-deoxyguanosine, 2′-deoxycytidine, and 2′-deoxythymidine,

L is selected from the group consisting of —(PO₂)—OCH₂—COH—CH₂—NH— and —(PO₂)—OCH₂—CH(CH₂COOH)—(CH₂)₄NH—.

### Formula II

5′ end of the chimeric oligonucleotide with a high affinity to protein:

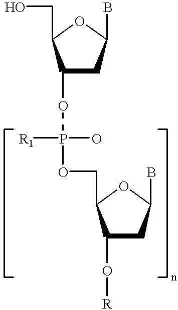

wherein,

n is at least 10 and not more than 20,

R₁ is selected from the group consisting of S⁻, CH₃, and O⁻,

B is selected from the group consisting of thymine, cytosine, adenine, and guanine,

### Formulas III/1, III/2, III/3

3′ end of the chimeric oligonucleotide with a high affinity to RNA:

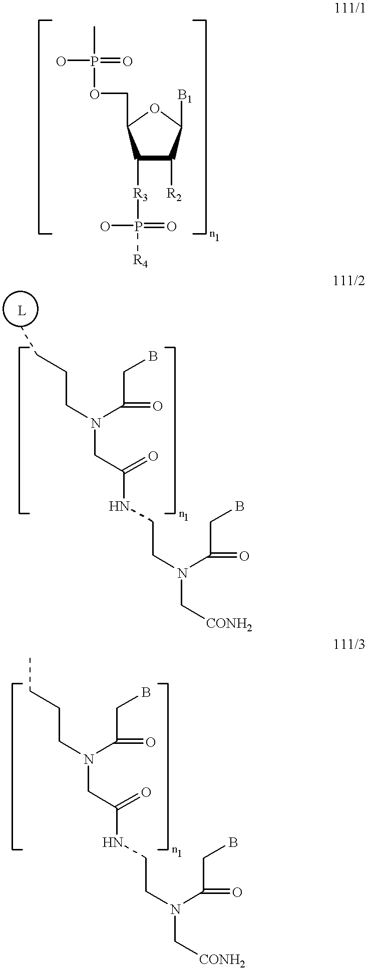

wherein

n₁ is at least 3 and not more than 17,

B₁ is selected from the group consisting of thymine, cytosine, adenine, guanine, 5-propyluracil, and 5-propylcytosine,

R₂ is selected from the group consisting of H, F, NH₂, O-alkyl (C₁-C₅), O-allyl, and O-methoxyethoxy,

R₃ is selected from the group consisting of NH and O, wherein if R₃ is NH, R₂ must not be selected from the group consisting of NH₂, O-alkyl (C₁-C₅), O-allyl, and O-methoxyethoxy,

R₄ is selected from the group consisting of 2′,3′-dideoxy-3′-fluoroguanosine, 2′,3′-dideoxy-3′-azidoguanosine, 2′,3′-dideoxy-3′-aminoguanosine, 2′,3′-epoxyguanosine, acyclovir, gancyclovir, 2′-deoxyadenosine, 2′-deoxyguanosine, 2′-deoxycytidine, and 2′-deoxythymidine,

L is selected from the group consisting of —(PO₂)—OCH₂—COH—CH₂—NH— and —(PO₂)—OCH₂—CH(CH₂COOH)—(CH₂)₄NH—.

Oligonucleotides modified by phosphorothioates or phosphoramidates and PNA are prepared by analogy with methods known as such (Chen, J. -K. et al. Nucleic Acids Res. (1995) 23, 2661-2668 and Lyer, R. P. et al., J. Org. Chem. (1990) 55, 4693-4699). Phosphorothioate is linked with PNA according to described methods, if necessary with using linkers (Uhlmann, E. et al. Ange. Chem. (1996) 18, 2793-2797). Modified guanosine derivates mentioned in R₄ are incorporated into the oligonucleotides in the form of their triphosphates with the terminal transferase.

The oligonucleotides have the following nucleotide sequences:

5′-TCAGATTAGTACTCGTCAGAGTTAGGGTTAG-3′ (SEQ ID No. 1)

5′-TCAGATTAGGACTGCTCAGAGTTAG-3′ (SEQ ID No. 2)

5′-TCAGATTAGTACTCGTCAGACAGTTAGGGTTAG-3′ (SEQ ID No. 3)

5′-TCAGATTAGTACTCGTCAGAGTTAGAGTTAG-3′ (SEQ ID No. 4)

5′-TCAGATTAGGACTGCTCAGAGUUAG-3′ (SEQ ID No. 5)

5′-TCAGATTAGGACTGCTCAGAUAGUUAG3′ (SEQ ID No. 6)

5′-TCAGATTAGGACTGCTCAGAGUUAGGGTTAGACAA-3′ (SEQ ID No. 7)

5′-TCAGATTAGGACTGCGTTAGGGTTAGACAA-3′ (SEQ ID No. 8)

5′-TCAGATTAGTACTCGTCAGA-O(PO₂)OCH₂CH(CH₂COOH—(CH₂))₄—NH-TAGGGTTAGACAA-3′ (SEQ ID No. 9)

5′ -TCAGATTAGTACTCGTCAGAGTTAGGGTTA-azidodeoxyguanosine-3′ (SEQ ID No. 10)

5′-AATCCTCCCCCAGTTCACCC-GTTAGGGT-3′ (SEQ ID No. 11)

5′-TCTCCCAGCGTGCGCCAT-GUUAGGGUUAG-3′ (SEQ ID No. 12)

5′-ATGTATGCTGTGGCT-n(L)-GTTAGG-3′ (SEQ ID No. 13)

5′-GTACTGCTCAGA-GTTAGGGTTAG-3′ (SEQ ID No. 14)

5′-GTACTGCTCAGA-GTTAGGGT-3′ (SEQ ID No. 15)

5′-GTACTGCTCAGA-GUUAGGGUUAG-3′ (SEQ ID No. 16)

5′-GTACTGCTCAGA-n(L)-GTTAGG-3′ (SEQ ID No. 17)

5′-GGCCAGCAGCTG-GUUAGGGUUAG-3′ (SEQ ID No. 18)

5′-TGCTCAGA-GUUAGGGUUAG-3′ (SEQ ID No. 19)

5′-TGCTCAGA-n(L)-GTTAGG-3′ (SEQ ID No. 20)

5′-TCAGACATATACTGCTCAGA-n(L)-TAGGGTTAGACAA-3′ (SEQ ID No. 21)

5′-ACT GCT CAG A-GTT AG-3′ (SEQ ID No. 22)

5′-ACT GCT CAG A-GUU AGG GUU AG-3′ (SEQ ID No. 23)

5′-ATA CTG CTC AGA-linker-GTT AGG GTT AG-3′ (SEQ ID No. 24)

5′-TTA GTA CTG CTC AGA-GTT AGG GTT AG-3′ (SEQ ID No. 25)

5′-TCA GAT TAG TAC TGC TCA GA-GTT AG-3′ (SEQ ID No. 26)

5′-TCA GAT TAG TAC TGC TCA GA-GTT AG-3′ (SEQ ID No. 27)

5′-ACT GCT CAG A-GTT AGGGTTAG-3′ (SEQ ID No. 28)

5′-TTAGGG-3′ (SEQ ID No. 29)

SEQ ID No. 1

Linkages between positions 1 to 20 are phosphorothioate linkages

Linkages between positions 20 to 26 are phosphordiester linkages

SEQ ID No. 2

Linkages between positions 1 to 20 are phosphorothioate linkages

SEQ ID No. 3

Linkages between positions 1 to 20 are phosphorothioate linkages

SEQ ID No. 4

Linkages between positions 1 to 20 are phosphorothioate linkages

SEQ ID No. 5

Linkages between positions 1 to 19 are phosphorothioate linkages

Linkages between positions 20 to 25 are ribose modified with 2′-OCH₃

SEQ ID No. 6

Linkages between positions 1 to 20 are phosphorothioate linkages

Linkages between positions 20 to 26 are ribose modified with 2′-OCH₃

SEQ ID No. 7

Linkages between positions 1 to 4 and positions 6 to 19 are phosphorothioate linkages

Linkages between positions 21 to 35 are ribose modified with 2′-OCH₃

SEQ ID No. 8

Linkages between positions 1 to 15 are phosphorothioate linkages

Linkages between positions 17 to 19 and 23 to 25 are phosphoramidate linkages

SEQ ID No. 9

Linkages between positions 1 to 16 are phosphorothioate linkages

X=3′-O(PO₂)OCH₂CH(CH₂COOH—(CH₂)₄—NH—

SEQ ID No. 10

Linkages between positions 1 to 20 are phosphorothioate linkages

g=3′ azidodeoxyguanosine

SEQ ID No. 11

linkages between positions 1 to 20 are phosphorothioates and

linkages between positions20 to 28 are N3′→N5′phosphoramidates

and position 28 is modified by a 3′-aminodeoxyribosyl residue

SEQ ID No. 12

linkages between positions 1 to 18 are phosphorothioates and

linkages between positions 18 to29 are phosphodiester linkages and

positions 19 to 29 carry 2′-OCH₃ modified ribosyl residues

SEQ ID No. 13

linkages between positions 1 to 15 are phosphorothioates and

linkages between positions 16 to 21 are modified by [N-(2-aminoethyl)glycine]methylene carbonyl residues and

the linker n (L) is —O(PO₂)—OCH₂—CH—(CH₂COOH)—(CH₂)₄—NH—

SEQ ID No. 14

linkages between positions 1 to 12 are phosphorothioates and

linkages between positions 12 to 23 are N3′→N5′phosphoramidates

and position 23 is modified by a 3′-aminodeoxribosyl residue

SEQ ID No. 15

linkages between positions 1 to 12 are phosphorothioates and

linkages between positions 12 to 20 are N3′→N5′phosphoramidates

and position 20 is modified by a 3′-aminodeoxribosyl residue

SEQ ID No. 16

linkages between positions 1 to 12 are phosphorothioates and

linkages between positions 12 to 23 are phosphodiester linkages and positions 13 to 23 carry 2′-OCH₃ modified ribosyl residues

SEQ ID No. 17

linkages between positions 1 to 12 are phosphorothioates and

linkages between positions 13 to 18 are modified by [N-(2-aminoethyl)glycine]methylene carbonyl residues and

the linker is —O(PO₂)—OCH₂—CH—(CH₂COOH)—(CH₂)₄—NH—

SEQ ID No. 18

linkages between positions 1 to 12 are phosphorothioates and

linkages between positions 12 to 23 are phosphodiester linkages and

positions 13 to 23 carry 2′-OCH₃ modified ribosyl residues

SEQ ID No. 19

linkages between positions 1 to 8 are phosphorothioates and

linkages between positions 8 to 19 are phosphodiester linkages and

positions 9 to 19 carry 2′-OCH₃ modified ribosyl residues

SEQ ID No. 20

linkages between positions 1 to 8 are phosphorothioates and

linkages between positions 8 to 14 are modified by [N-(2-aminoethyl) glycine]methylene carbonyl residues and

the linker is —O(PO₂)—OCH₂—CH—(CH₂COOH)—(CH₂)₄—NH—

SEQ ID No. 21

linkages between positions 1 to 20 are phosphorothioates and

linkages between positions 20 to 33 are modified by [N-(2-aminoethyl)glycine]methylene carbonyl residues and

the linker is —O(PO₂)—OCH₂—CH—(CH₂COOH)—(CH₂)₄—NH—

SEQ ID No. 22

linkages between positions 1 to 10 are phosphorothioates and

linkages between positions 10 to 15 are N3′→N5′phosphoramidates

and position 15 is modified by a 3′-aminodeoxribosyl residue

SEQ ID No. 23

linkages between positions 1 to 10 are phosphorothioates and

linkages between positions 10 to21 are phosphodiester linkages and

positions 11 to 21 carry 2′-OCH₃ modified ribosyl residues

SEQ ID No. 24

linkages between positions 1 to 12 are phosphorothioates and

linkages between positions 13 to 23 are modified by [N-(2-aminoethyl)glycine]methylene carbonyl residues and

the linker is —O(PO₂)—OCH₂—CH—(CH₂COOH)—(CH₂)₄—NH—

SEQ ID No. 25

linkages between positions 1 to 15 are phosphorothioates and

linkages between positions 15 to 26 are N3′→N5′phosphoramidates

and position 26 is modified by a 3′-aminodeoxyribosyl residue

SEQ ID No. 26

linkages between positions 1 to 20 are phosphorothioates

linkages between positions 20 to 25 are N3′→N5′phosphoramidates

and position 25 is modified by a 3′-aminodeoxyribosyl residue

SEQ ID No. 27

linkages between positions 1 to 19 are phosphorothioates

linkages between positions 20 to 24 are N3′→N5′phosphoramidates

and position 25 is modified by a 3′-aminodeoxyribosyl residue

SEQ ID No. 28

linkages between position 1 to 10 are phosphorothioates and

linkages between positions 10 to 21 are N3′→N5′phosphoramidates

and position 21 is modified by a 3′-aminodeoxyribosyl residue

The oligonucleotides according to the invention of the general formula I block both binding sites of the telomeric DNA on the enzyme, at the same time, thus being highly efficient and highly selective inhibitors of telomerase.

If necessary, oligonucleotides were applied to tumor cells as complexes with cationic liposomes or other suitable means of transport, resulting here in a critical shortening of the telomere DNA and finally in the death of the cell after permanently inhibiting telomerase.

The chimeric oligonucleotides according to the invention are used for preparing pharmaceutical administrative forms by formulating them with pharmaceutical additives and auxiliary and supporting agents.

The medicaments thus produced are highly efficient cancerostatic agents.

Hereinafter, the invention will be explained in greater detail by an example of execution.

### EXAMPLE OF EXECUTION

The oligonucleotide no. 8 was prepared in a DNA synthesizer of the company Applied Biosystem, model 391, on a 1 μmol scale, according to the protocols of the equipment manufacturer, with using cyanoethyl phosphoamidites. The phosphorothioate bonds were formed by means of tetraethyl thiuram disulfide (Lit.: H. Vu and B. L. Hirschbein, Tetrahedron Lett. (1991) 32, 3005-3008). The decisive step to an automated synthesis of the phosphoramide bonds of the oligomer consists in the reaction of the 5′-(n,n-diisopropylamino-2-cyanoethyl)-phosphoamidite-3′-(trityl)amino-2′,3′-dideoxythymidine monomer (Lit.: McCardy, S. N. et al. Tetrahedron Lett. (1997) 2, 207-210) with the 3′-OH-nucleotide bound to the solid phase or a 3′-aminonucleotide of the growing nucleotide chain. The resulting phosphoamidite was oxidised to form stable phosphoarmidate. After removing the basic protective groups by means of ammonia the oligomere was purified by means of denaturating gel electrophoresis. The oligonucleotide no. 8 was desalted (NAP 10, Phamacia) and lyophylized.

Cells of the human tumour cell line HL60 were lysed and a 1000-cell equivalent of this extract was used in the TRAP assay (Telomeric repeat amplification protocol) described by Piatyzek et al. in 1995 for the determination of the efficiency of the two chimeric oligonucleotides nos. 5 and 8 to inhibit telomerase activity. In principle, a radioactively lebeld primer was thereby elongated by the activity of telomerase and the telomerase product formed after PCR amplification and gel electrophoresis was quantitatively evaluated by phosphorus imaging. The oligonucleotides nos. 5 and 8 are in a position to strongly inhibit the activity of telomerase. An inhibition of the telomerase activity by 50% is reached by oligonucleotide no. 5 at 0.5 nM and by oligonucleotide no. 8 at 1 nM.

Cells of the human cell line HL60 were lysed and a 1000-cell equivalent of this extract was used to estimate the telomerase activity in the TRAP assay (telomeric repeat amplification protocol; as described by Piatyzek et al., 1995). In principle, a radioactive (³²-P-phosphate) labeled primer was elongated by the activity of telomerase and the products formed were amplified by PCR, separated by gel electrophoresis and than quantitatively evaluated by ³²P-imaging. Oligonucleotides were added to the assay and the concentrations were estimated required for a 50% inhibition of telomerase activity (IC₅₀ values).

Table 1 summarizes the results (IC₅₀ values) for some representative oligonucleotides.

TABLE 1
Concentrations required for a 50% inhibition of telomerase activity
(IC₅₀) in HL60 cell-lysate by chimeric oligomers

| | IC₅₀ |
|---|---|
| | values |
| 5′-ACT GCT CAG A-GTT AG-3′ (no. 22) | 3.5 nM |

linkages between positions 1 to 10 are phosphorothioates and
linkages between positions 10 to 15 are N3′→N5′ phosphoramidates
and position 15 is modified by a 3′-aminodeoxribosyl residue

| | |
|---|---|
| 5′-ACT GCT CAG A-GUU AGG GUU AG-3′ (no. 23) | 1.8 nM |

linkages between positions 1 to 10 are phosphorothioates and
linkages between positions 10 to 21 are phosphodiester linkages and
positions 11 to 21 carry 2′-OCH₃ modified ribosyl residues

| | |
|---|---|
| 5′-ATA CTG CTC AGA-linker-GTT AGG GTT AG-3′ | 1.6 nM |
| (no. 24) | |

linkages between positions 1 to 12 are phosphorothioates and linkages
between positions 14 to 24 are modified by [N-(2-aminoethyl)
glycine]methylene carbonyl residues and position 13 = linker is
—O(PO₂)—OCH₂—CH—(CH₂ COOH)—(CH₂)₄—NH—

| | |
|---|---|
| 5′-TTA GTA CTG CTC AGA-GTT AGG GTT AG-3′ | 1.4 nM |
| (no. 25) | |

linkages between positions 1 to 15 are phosphorothioates and
linkages between positions 15 to 26 are N3′→N5′ phosphoramidates
and position 26 is modified by a 3′-aminodeoxyribosyl residue

| | |
|---|---|
| 5′-TCA GAT TAG GAC TGC TCA GA-GUUAG-3′ (no. 5) | 0.5 nM |

linkages between positions 1 to 20 are phosphorothioates and
linkages between positions 20 to 25 are phosphodiesters and
positions 21 to 25 carry 2′-OCH₃ modified ribosyl residues

| | |
|---|---|
| 5′-TCA GAT TAG TAC TGC TCA GA-GTT AG-3′ (no. 26) | 1.5 nM |

linkages between positions 1 to 20 are phosphorothioates
linkages between positions 20 to 25 are N3′→N5′ phosphoramidates
and position 25 is modified by a 3′-aminodeoxyribosyl residue

For testing at cellular level the human glioblastoma cell line U87 was plated at 90000 cells/well in 24 well plates in Eagle's Minimal Essential Medium (EMEM) supplemented with 5% Basal Medium Supplement, 2 mM GlutaMax™ (Life Technologies) 10% fetal calf serum (FCS), 100 U/ml penicillin, 100 μg/ml streptomycin at 37° C. in a 5% CO₂ atmosphere.

For transient transfection of oligonucleotides the adherent U87 cells were washed with EMEM incubated for 4 h (at 37° C.; 5% CO₂/air) with Lipofectin/oligonucleotide complex in 0.4 ml EMEM supplemented only by 3% fetal calf serum.

To prepare the complex each of the component, the oligonucleotide (up to 10 μM) as well as the Lipofectin (2.5/5 μg; Life Technologies, Gaithersburg, Md.) was diluted in 50 μl of serum-free EMEM according to manufacture's instruction. Both solutions were mixed and kept at room temperature for 15 min and overlayed onto cells (covered with 0.3 ml EMEM/4% FCS). After removing of the transfecting mixture (4 h later) the cells were washed (1×0.3 ml PBS, 3×0.25 ml serum containing medium) and incubated for 3 days in 1 ml complete EMEM. Cells were washed (2×0.25 ml PBS) trypsinized, pelleted, washed again (2×0.25 ml PBS, 1×0.5 ml PBS), counted, lysed and stored as described. The lysates were used for the TRAP assay as described for the HL60 cell-lysates. Table 2 demonstrates some of the results obtained.

TABLE 2
Concentrations required for a 50% inhibition of telomerase activity in U87
glioblas-toma cells (ID₅₀), 3 days after transfection with chimeric oligomers complexed
with lipofectin. The primary cytotoxicity of these oligomers is given by the %
inhibition of cell proliferation at 1 μM.

| | | % inhibition of cell |
|---|---|---|
| | IC₅₀-values | proliferation at 1 μM |
| 5′-ACT GCT CAG A-GTT AG-3′ (no. 22) | >>1.20 μM | 20 |

linkages between position 1 to 10 are phosphorothioates and
linkages between positions 10 to 15 are N3′→N5′ phosphoramidates
and position 15 is modified by a 3′-aminodeoxyribosyl residue

| | | |
|---|---|---|
| 5′-ACT GCT CAG A-GUU AGG GUU AG-3′ (no. 23) | 0.6 μM | 27 |

linkages between positions 1 to 10 are phosphorothioates and
linkages between positions 10 to 21 are phosphodiesters and
positions 11 to 21 carry 2′-OCH₃ modified ribosyl residues

| | | |
|---|---|---|
| 5′-ATA CTG CTC AGA-linker-GTT AGG GTT AG-3′ (no. 24) | 0.05 μM | 41 |

linkages between positions 1 to 12 are phosphorothioates and
linkages between positions 14 to 24 are modified by [N-(2-aminoethyl)
glycine]methylene carbonyl linkages and position 13
linker is —O(PO₂)—OCH₂—CH—(CH₂ COOH)—(CH₂)₄—NH—

| | | |
|---|---|---|
| 5′-TTA GTA CTG CTC AGA-GTT AGG GTT AG-3′ (no. 25) | 0.37 μM | 39 |

linkages between positions 1 to 15 are phosphorothioates and
linkages between positions 15 to 26 are N3′→N5′ phosphoramidates
and position 26 is modified by a 3′-aminodeoxribosyl residue

| | | |
|---|---|---|
| 5′-TCA GAT TAG GAC TGC TCA GA-GTT AGG GTT AG-3′ (no. 27) | 0.08 μM | 62 |

linkages between positions 1 to 20 are phosphorothioates and
linkages between positions 20 to 31 are N3′→N5′ phosphoramidates
and position 31 is modified by a 3′-aminodeoxyribosyl residue

| | | |
|---|---|---|
| 5′-TCA GAT TAG GAC TGC TCA GA-GUUAG-3′ (no. 5) | 0.4 μM | 56 |

linkages between positions 1 to 20 are phosphorothioates and
linkages between positions 20 to 25 are phosphodiesters and
positions 21 to 25 carry 2′-OCH₃ modified ribosyl residues

| | | |
|---|---|---|
| 5′-TCA GAT TAG TAC TGC TCA GA-GTT AG-3′ (no. 26) | 0.56 μM | 58 |

linkages between positions 1 to 20 are phosphorothioates
linkages between positions 20 to 25 are N3′→N5′ phosphoramidates
and position 25 is modified by a 3′-aminodeoxyribosyl residue

For in vivo experiments U-87 tumors were implanted subcutaneously into the flank region of six-week-old athymic BALB/c nu/nu mice. After 2-3 weeks, groups of 6 mice were injected once intravenously either with 8 mg/kg of one the oligonucleotides (see below) dissolved in PBS or with PBS alone. Three day later the tumors were removed, washed and treated with collagenase and hyaloronidase. The cell suspensions obtained were treated with magnetobeads coated with mouse antibodies against human HLA-ABC antigen as well as with a secondary anti-mouse antibody to separate the U87 cells. These isolated U87 cells were lysed and the telomerase activity was estimated for each tumor as described. The data given in Table 3 represent mean values of telomerase activity in treated mice related to the telomerase activity of tumors of untreated animals.

TABLE 3
Inhibition of telomerase in U87 tumors, 3 days after a single
intravenous injection of nude mice with 8 mg/kg of the described
oligonucleotides. The % inhibition is related to the telomerase
activity in U87 tumors of untreated nude mice.

| | Inhibition of telomerase |
|---|---|
| | activity in U87 tumors in |
| | nude mice treated with |
| | oligonucleotides |
| 5′-GTACTGCTCAGA-GUUAGGGUUAG-3′ | 54% |
| (no. 16) | |

linkages between positions 1-12 phosphorothioates and
linkages between positions 12-20 phosphodiesters and
linkages between positions 20 to 23 are phosphorothioates,
positions 13-23 carry 2′-OCH₃ modified ribosyl residues

| | |
|---|---|
| 5′-ACT GCT CAG A-GTT AGGGTTAG-3′ | 71% |
| (no. 28) | |

linkages between position 1 to 10 are phosphorothioates and
linkages between positions 10 to 21 are N3′→N5′ phosphoramidates
and position 21 is modified by a 3′-aminodeoxyribosyl residue

1. Chimeric oligonucleotides of a general formula I for binding telomerase, comprising, 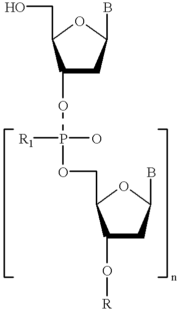
wherein R is selected from the group consisting of 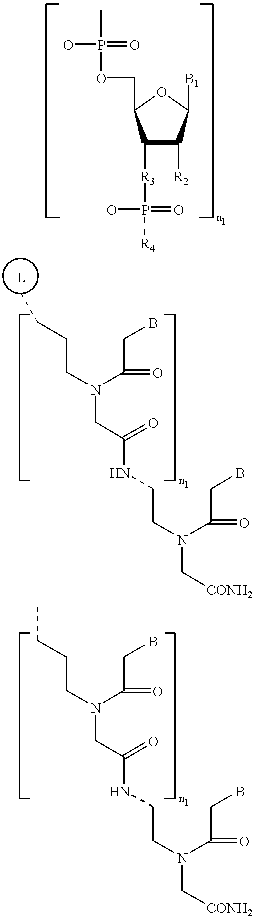
wherein
n is at least 10 and not more than 20,
R₁ is selected from the group consisting of S⁻, CH₃, and O⁻,
B is selected from the group consisting of thymine, cytosine, adenine, and guanine,
n₁ is at least 3 and not more than 17,
B₁ is selected from the group consisting of thymine, cytosine, adenine, guanine, 5-propyluracil, and 5-propylcytosine,
R₂ is selected from the group consisting of H, F, NH₂, O-alkyl (C₁-C₅), O-allyl, and O-methoxyethoxy,
R₃ is selected from the group consisting of NH and O, wherein if R₃ is NH, R₂ must not be selected from the group consisting of NH₂, O-alkyl (C₁-C₅), O-allyl, and O-methoxyethoxy,
R₄ is selected from the group consisting of 2′,3′-dideoxy-3′-fluoroguanosine, 2′,3′-dideoxy-3′-azidoguanosine, 2′,3′-dideoxy-3′-aminoguanosine, 2′,3′-epoxyguanosine, acyclovir, gancyclovir, 2′-deoxyadenosine, 2′-deoxyguanosine, 2′-deoxycytidine, and 2′-deoxythymidine,
L is selected from the group consisting of —(PO₂)—OCH₂—COH—CH₂—NH— and —(PO₂)—OCH₂—CH(CH₂COOH)—(CH₂)₄NH—.

2. The oligonucleotides according to claim 1, wherein R is

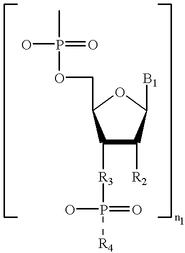

3. The oligonucleotides according to claim 1, wherein R is

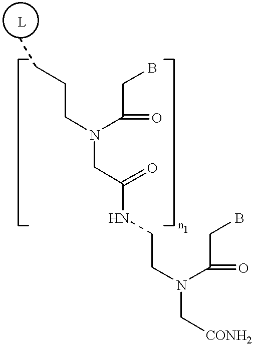

4. The oligonucleotides according to claim 1, wherein R is

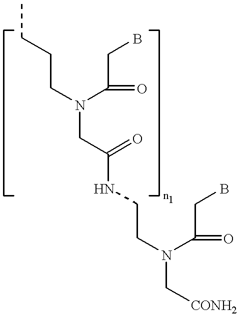

5. The oligonucleotides according to claim 1, wherein R1 to R4 and B and B1 vary from a nucleotide unit to another nucleotide unit.

6. The oligonucleotides according to claim 1, wherein the oligonucleotides having a nucleotide sequence is selected from the group consisting of
5′-TCAGATTAGTACTCGTCAGAGTTAGGGTTAG-3′ (SEQ ID No. 1)
5′-TCAGATTAGGACTGCTCAGAGTTAG-3′ (SEQ ID No. 2)
5′-TCAGATTAGTACTCGTCAGACAGTTAGGGTTAG-3′ (SEQ ID No. 3)
5′-TCAGATTAGTACTCGTCAGAGTTAGAGTTAG-3′ (SEQ ID No. 4)
5′-TCAGATTAGGACTGCTCAGAGUUAG-3′ (SEQ ID No. 5)
5′-TCAGATTAGGACTGCTCAGAUAGUUAG3′ (SEQ ID No. 6)
5′-TCAGATTAGGACTGCTCAGAGUUAGGGTTAGACAA-3′ (SEQ ID No. 7)
5′-TCAGATTAGGACTGCGTTAGGGTTAGACAA-3′ (SEQ ID No. 8)
5′-TCAGATTAGTACTCGTCAGA-O(PO₂)OCH₂CH(CH₂COOH—(CH₂))₄—NH-TAGGGTTAGACAA-3′ (SEQ ID No. 9)
5′-TCAGATTAGTACTCGTCAGAGTTAGGGTTA-azidodeoxyguanosine-3′ (SEQ ID No. 10)
5′-AATCCTCCCCCAGTTCACCC-GTTAGGGT-3′ (SEQ ID No. 11)
5′-TCTCCCAGCGTGCGCCAT-GUUAGGGUUAG-3′ (SEQ ID No. 12)
5′-ATGTATGCTGTGGCT-n(L)-GTTAGG-3′ (SEQ ID No. 13)
5′-GTACTGCTCAGA-GTTAGGGTTAG-3′ (SEQ ID No. 14)
5′-GTACTGCTCAGA-GTTAGGGT-3′ (SEQ ID No. 15)
5′-GTACTGCTCAGA-GUUAGGGUUAG-3′ (SEQ ID No. 16)
5′-GTACTGCTCAGA-n(L)-GTTAGG-3′ (SEQ ID No. 17)
5′-GGCCAGCAGCTG-GUUAGGGUUAG-3′ (SEQ ID No. 18)
5′-TGCTCAGA-GUUAGGGUUAG-3′ (SEQ ID No. 19)
5′-TGCTCAGA-n(L)-GTTAGG-3′ (SEQ ID No. 20)
5′-TCAGACATATACTGCTCAGA-n(L)-TAGGGTTAGACAA-3′ (SEQ ID No. 21)
5′-ACT GCT CAG A-GTT AG-3′ (SEQ ID No. 22)
5′-ACT GCT CAG A-GUU AGG GUU AG-3′ (SEQ ID No. 23)
5′-ATA CTG CTC AGA-linker-GTT AGG GTT AG-3′ (SEQ ID No. 24)
5′-TTA GTA CTG CTC AGA-GTT AGG GTT AG-3′ (SEQ ID No. 25)
5′-TCA GAT TAG TAC TGC TCA GA-GTT AG-3′ (SEQ ID No. 26)
5′-TCA GAT TAG TAC TGC TCA GA-GTT AG-3′ (SEQ ID No. 27)
5′-ACT GCT CAG A-GTT AGGGTTAG-3′ (SEQ ID No. 28)
5′-TTAGGG-3′ (SEQ ID No. 29).

7. A method of inhibiting telomerase activity, comprising the administering of chimeric oligonucleotides to a human tumor cell line.

8. A method of in vivo treatment of tumours, comprising the administering of chimeric oligonucleotides in a flank region.


SEQUENCE LISTING (29 sequences)

SEQ ID NO: 1 - DNA, 31 bp, Artificial Sequence
  Description of Artificial Sequence oligonucleotide,  linkages between positions 1 to 20 are phosphorothioates, linkages between positions 20 to 31 are phosphodiesters
tcagattagtactcgtcagagttagggttag

SEQ ID NO: 2 - DNA, 25 bp, Artificial Sequence
  Description of Artificial Sequence oligonucleotide, linkages between positions 1 to 20 are phosphorothioates,  linkages between positions 20 to 25 are phosphodiesters
tcagattaggactgctcagagttag

SEQ ID NO: 3 - DNA, 33 bp, Artificial Sequence
  Description of Artificial Sequence oligonucleotide,  linkages between positions 1 to 20 are phosphorothioates,  linkages between positions 20 to 33 are phosphodiesters
tcagattagtactcgtcagacagttagggttag

SEQ ID NO: 4 - DNA, 31 bp, Artificial Sequence
  Description of Artificial Sequence oligonucleotide,  linkages between  positions 1 to 20 are phosphorothioates,  linkages between positions 20 to 31 are phosphodiesters
tcagattagtactcgtcagagttagagttag

SEQ ID NO: 5 - DNA, 25 bp, Artificial Sequence
  Description of Combined DNA/RNA Molecule oligonucleotide,  linkages between  positions 1 to 19  are phosphorothioates,  linkages between positions  19 to 25 are phosphodiesters
tcagattaggactgctcagaguuag

SEQ ID NO: 6 - DNA, 27 bp, Artificial Sequence
  Description of Combined DNA/RNA Molecule oligonucleotide,linkages between positions 1 to 20 are phosphorothioates,  linkages between positions 20  to 27 are phosphodiesters,
tcagattaggactgctcagauaguuag

SEQ ID NO: 7 - DNA, 35 bp, Artificial Sequence
  Description of Combined DNA/RNA Molecule oligonucleotide,  linkages between positions 1 to 19 are  phosphorothioates,  linkages between positions 19 to 35 are phosphodiesters,
tcagattaggactgctcagaguuagggttagacaa

SEQ ID NO: 8 - DNA, 30 bp, Artificial Sequence
  Description of Artificial Sequence oligonucleotide, linkages between positions 1 to 15 are phosphorothioates,  linkages between positions 15 to 17, 19 to 23 and 25 to 30 are phosphodiesters,
tcagattaggactgcgttagggttagacaa

SEQ ID NO: 9 - DNA, 34 bp, Artificial Sequence
  Description of Combined DNA/RNA Molecule oligonucleotide, linkages between  positions 1 to 15 are phosphorothioates,  linkages between positions 15 to 20 are phosphodiesters,
tcagattagtactcgtcagantagggttagacaa

SEQ ID NO: 10 - DNA, 31 bp, Artificial Sequence; identical to SEQ ID NO: 1
  Description of Artificial Sequence oligonucleotide, linkages between  positions 1 to 20 are phosphorothioates,  linkages between positions 20 to 31 are phosphodiesters,

SEQ ID NO: 11 - DNA, 28 bp, Artificial Sequence
  Description of Artificial Sequence oligonucleotides, linkages between positions   1 to 20  are phosphorothioate, linkages between positions 20 to 28 are N3′ to  N5′ phosphoramidates
aatcctcccccagttcacccgttagggt

SEQ ID NO: 12 - DNA, 29 bp, Artificial Sequence
  Description of Combined DNA/RNA Molecule oligonucleotide
tctcccagcgtgcgccatguuaggguuag

SEQ ID NO: 13 - DNA, 25 bp, Artificial Sequence
  Description of Artificial Sequence oligonucleotides, linkages between positions   1 to 15  are phosphorothioates,
atgtatgctgtggctngttaggagg

SEQ ID NO: 14 - DNA, 23 bp, Artificial Sequence
  Description of Artificial Sequence oligonucleotides, linkages between positions 1 to 12 are phosphorothioates, linkages between positions 12 to 23 are N3′ to N5′ phosphoramidates
gtactgctcagagttagggttag

SEQ ID NO: 15 - DNA, 20 bp, Artificial Sequence
  Description of Artificial Sequence oligonucleotides, linkages between positions   1 to 12 are phosphorothioates,  linkages between positions 12 to 20 are N3′ to N5′ phosphoramidates
gtactgctcagagttagggt

SEQ ID NO: 16 - DNA, 23 bp, Artificial Sequence
  Description of Combined DNA/RNA Molecule oligonucleotide
gtactgctcagaguuaggguuag

SEQ ID NO: 17 - DNA, 19 bp, Artificial Sequence
  Description of Artificial Sequence oligonucleotides, linkages between positions  1 to 12 are phosphorothioates,
gtactgctcagangttagg

SEQ ID NO: 18 - DNA, 23 bp, Artificial Sequence
  Description of Combined DNA/RNA Molecule oligonucleotide
ggccagcagctgguuaggguuag

SEQ ID NO: 19 - DNA, 19 bp, Artificial Sequence
  Description of Combined DNA/RNA Molecule oligonucleotide
tgctcagaguuaggguuag

SEQ ID NO: 20 - DNA, 15 bp, Artificial Sequence
  Description of Artificial Sequence oligonucleotides, linkages between positions 1 to 8 are phosphorothioates,
tgctcagangttagg

SEQ ID NO: 21 - DNA, 34 bp, Artificial Sequence
  Description of Artificial Sequence oligonucleotides, linkages between positions   1 to 20 are phosphorothioates,
tcagacatatactgctcagantagggttagacaa

SEQ ID NO: 22 - DNA, 15 bp, Artificial Sequence
  Description of Artificial Sequence oligonucleotides, linkages between positions   1 to 10 are phosphorothioates, linkages between positions 10 to 15 are N3′ to N5′ phosphoramidates
actgctcagagttag

SEQ ID NO: 23 - DNA, 21 bp, Artificial Sequence
  Description of Combined DNA/RNA Molecule oligonucleotide
actgctcagaguuaggguuag

SEQ ID NO: 24 - DNA, 24 bp, Artificial Sequence
  Description of Artificial Sequence oligonucleotides, linkages between positions 1 to 12 are phosphorothioates,
atactgctcagangttagggttag

SEQ ID NO: 25 - DNA, 26 bp, Artificial Sequence
  Description of Artificial Sequence oligonucleotides, linkages between positions   1 to 15 are phosphorothioates,   linkages between positions 15 to 26 are N3′ to N5′ phosphoramidates
ttagtactgctcagagttagggttag

SEQ ID NO: 26 - DNA, 25 bp, Artificial Sequence
  Description of Artificial Sequence oligonucleotides, linkages between positions   1 to 20 are phosphorothioates,  linkages between positions 20 to 25 are N3′ to N5′ phosphoramidates
tcagattagtactgctcagagttag

SEQ ID NO: 27 - DNA, 31 bp, Artificial Sequence
  Description of Artificial Sequence oligonucleotides, linkages between positions 1 to 20 are phosphorothioates, linkages between positions 20 to 31 are N3′ to N5′ phosphoramidates
tcagattagtactgctcagagttagggttag

SEQ ID NO: 28 - DNA, 21 bp, Artificial Sequence
  Description of Artificial Sequence oligonucleotides, linkages between positions   1 to 10 are phosphorothioates,  linkages between positions 10 to 21 are N3′ to N5′ phosphoramidates
actgctcagagttagggttag

SEQ ID NO: 29 - DNA, 6 bp, Artificial Sequence
  Description of Artificial Sequence  telomeric DNA of man
ttaggg